(19) United States
(12) Patent Application Publication
(10) Pub. No.: US 2005/0010068 A1
(43) Pub. Date: Jan. 13, 2005

(54) Use of microencapsulated transition metal reagents for reactions in supercritical fluids

(76) Inventors: Andrew Bruce Holmes, Cambridge (GB); Chandrashekar Ramarao, Hyderabad (IN); Richard Spencer Gordon, Cambridgeshire (GB); Steven Victor Ley, Cambridge (GB); Tessa Rachel Early, Cambridge (GB)

Correspondence Address: MORGAN LEWIS & BOCKIUS LLP, 1111 PENNSYLVANIA AVENUE NW, WASHINGTON, DC, 20004, US

(21) Appl. No.: 10/496,662

(22) PCT Filed: Nov. 29, 2002
(86) PCT No.: PCT/GB02/05419

(30) Foreign Application Priority Data
Dec. 1, 2001 (GB) 0128839.8

(51) Int. Cl. C07C 39/17

(52) U.S. Cl. 568/713

(57) ABSTRACT

There is provided processes for metal mediated reactions, particularly cross coupling and carbometallation reactions, wherein the metal is present as a catalyst system comprising a catalyst microencapsulated within a permeable polymer microcapsule shell and the reaction is carried out under super-critical or near super-critical conditions. Preferred metal-mediated cross coupling reactions include Heck, Suzuki, Sonogashira and Stille reactions. Preferred carbometallation reactions include hydroformylations.

This invention relates to the use of microencapsulated transition metal reagents for reactions, particularly metal mediated cross coupling reactions and carbometallation reactions, in supercritical fluids.

There has been considerable interest in the application of supercritical carbon dioxide (sc CO₂) as a solvent for chemical synthesis as a result of its unique physical properties and its environmentally friendly nature (R. S. Oakes, A. A. Clifford and C. M. Rayner, J. Chem. Soc., Perkin Trans. 1, 2001, 917-941; J. A. Darr and M. Poliakoff, Chem. Rev., 1999, 99, 495; P. G. Jessop and W. Leitner, Chemical Synthesis Using Supercritical Fluids, Wiley-VCH, Weinhein, 1999; F. Liu, M. B. Abrams, R. T. Baker and W. Tumas, Chem. Commun., 2001, 433; M. A. Carroll and A. B. Holmes, Chem. Commun., 1998, 1395; D. K. Morita, D. R. Pesiri, S. A. David, W. H. Glaze and W. Tumas, Chem. Commun., 1998, 1397; N. Shezad, R. S. Oakes, A. A. Clifford and C. M. Rayner, Tetrahedron Lett., 1999, 40, 2221; T. Osswald, S. Schneider, S. Wang and W. Bannwarth, Tetrahedron Lett., 2001, 42, 2965. Homogeneous cross-coupling reactions in sc CO₂ have been reported. Recently, we and others described the application of fluorine free coupling reactions in sc CO₂. In our report we detailed the first examples of solid supported reactions, in sc CO₂, in which the reactants were anchored to a polystyrene resin (T. R. Early, R. S. Gordon, M. A. Carroll, A. B. Holmes, R E. Shute and 1. F. McConvey, Chem Commun., 2001, 1966).

Metal-catalysed processes are extremely common in the synthesis of small organic molecules for the pharmaceutical industry as well as for agrochemicals, flavours, fragrances and specialist consumer products.

PCT/GB 99/000294 discloses the use of CO₂-solubilising perfluorinated phosphine derivatives to solubilise palladium(II) and palladium(0) complexes to mediate various organometallic cross coupling reactions. Specific reactions of interest included the Heck reaction (the palladium-mediated addition of an aryl or vinyl halide to an alkene with regeneration of the double bond in the original alkene partner; see Palladium reagents in organic synthesis, R. F. Heck, Academic Press, Orlando, 1985; Heck, R. F., Org. React, 1982, 27, 345; Beletskaya, I.; Cheprakov, A. Chem. Rev., 2000, 100, 309), the Suzuki reaction, [the palladium(0)-mediated cross coupling of an organoboronate or boronic acid derivative with a functionalised unsaturated molecule such as an aryl or vinyl halide or an aryl or vinyl trifluoroalkanesulfonate (Suzuki, A. in Metal-catalysed Cross-coupling reactions, eds. Diederich, F. and Stang, P. J., Wiley-VCH, Weinheim, 1997)]. Other coupling reactions of interest are the Stille coupling of an organostannane with an aryl or vinyl halide or trifluoroalkanesulfonate (see J. K. Stille, Angew. Chem. Int Ed., 1986, 25, 508) and the Sonogashira reaction involving the coupling of an acetylide with aryl or vinyl halides in the presence of a non-nucleophilic base and a catalytic quantity of a copper(I) salt (see K. Sonogashira, Y. Toda and N. Hagihara, Tetrahedron Left., 1975, 4467).

Recently some of the inventors of the present patent application disclosed (GB patent application no. 0117037.2) the preparation of a novel polyurea prepared by interfacial polymerisation techniques which enabled encapsulated transition metal derivatives to be prepared. Specifically disclosed was supported palladium catalysts prepared using palladium(II) acetate and osmium tetroxide catalysts. Supported reagents have been used in a variety of chemical transformations (S. V. Ley, I. R. Baxendale, R. N. Bream, P. S. Jackson, A. G. Leach, D. A. Longbottom, M. Nesi, J. S. Scott, R. I. Storer and S. J. Taylor, J. Chem. Soc., Perkin Trans. 1, 2000, 3815).

There have been few disclosures of the use of supported reagents in supercritical carbon dioxide. Pd/C has been used to promote the Heck reaction in sc CO₂, but with rather long reactions times (S. Cacchi, G. Fabrizi, F. Gasparrini and C. Villani, Synlett, 1999, 345) and recently a dendrimer-supported Pd reagent has been described (L. K. Yeung, C. T. Lee, K. P. Johnston and R. M. Crooks, Chem. Commun., 2001, 2290. For a recent example of nucleophilic displacement reactions using silica-supported phase transfer agents in sc CO₂ see J. DeSimone, M. Selva and P. Tundo, J. Org. Chem.; 2000, 66, 4047.

In this disclosure the use of encapsulated palladium prepared as described in GB application no. 0117037.2 is surprisingly found to be superior to conventional palladium catalysts in sc CO₂. Encapsulated palladium leads to enhanced yields in metal-mediated cross coupling reactions (Heck, Suzuki, Sonogashira, Stille) and is useful for other metal mediated cross coupling and carbometallation reactions (e.g. hydroformylation).

According to a first aspect of the present invention there is provided a process for metal mediated reactions, particularly cross coupling and carbometallation reactions, wherein the metal is present as a catalyst system comprising a catalyst microencapsulated within a permeable polymer microcapsule shell and the reaction is carried out under super-critical or near super-critical conditions.

The term “encapsulation” has different connotations depending on the application area. Microencapsulation in the present context describes the containment of a finely divided solid or liquid in a polymeric micro-particle, where milling or grinding a larger mass has not made the micro-particle. The term ‘monolithic’ or ‘matrix’ describes a particle having a finely divided solid or liquid distributed throughout a ‘solid’ or amorphous polymeric bead, while the term ‘reservoir’ describes a particle where the finely divided solid or liquid is contained within an inner cavity bound by an integral outer polymer shell. Thus as used herein the term “microencapsulated within a permeable polymer microcapsule shell” indicates that the polymer shell containing the catalyst is itself in the form of a microcapsule, formed for example by one of the techniques described in greater detail below. A microcapsule formed by such techniques will be generally spherical or collapsed spherical and have a mean diameter of from 1 to 1000 microns, preferably from 25 to 500 microns and especially from 50 to 300 microns. The polymer microcapsule shell is permeable to the extent that the reaction medium being catalysed is capable of contacting the encapsulated catalyst.

Various processes for microencapsulating material are available. These processes can be divided into three broad categories (a) physical, (b) phase separation and (c) interfacial reaction methods. In the physical methods category, microcapsule wall material and core particles are physically brought together and the wall material flows around the core particle to form the microcapsule. In the phase separation category, microcapsules are formed by emulsifying or dispersing the core material in an immiscible continuous phase in which the wall material is dissolved and caused to physically separate from the continuous phase, such as by coacervation, and deposit around the core particles. In the interfacial reaction category, the core material is emulsified or dispersed in an immiscible continuous phase, and then an interfacial polymerization reaction is caused to take place at the surface of the core particles thereby forming microcapsules.

The above processes vary in utility. Physical methods, such as spray drying, spray chilling and humidized bed spray coating, have limited utility for the microencapsulation of products because of volatility losses and pollution control problems associated with evaporation of solvent or cooling, and because under most conditions not all of the product is encapsulated nor do all of the polymer particles contain product cores. Phase separation techniques suffer from process control and product loading limitations. It may be difficult to achieve reproducible phase separation conditions, and it may be difficult to assure that the phase-separated polymer will preferentially wet the core droplets.

Interfacial polymerisation reaction methods are therefore preferred for encapsulation of the catalyst within the polymer microcapsule shell.

Thus according to a further aspect of the present invention there is provided a process for metal mediated reactions, particularly cross coupling and carbometallation reactions, wherein the metal is present as a catalyst system comprising a catalyst microencapsulated within a permeable polymer microcapsule shell wherein the microcapsule shell is formed by interfacial polymerisation and the reaction is carried out under super-critical or near super-critical conditions.

There are various types of interfacial polymerisation techniques but all involve reaction at the interface of a dispersed phase and a continuous phase in an emulsion system. Typically the dispersed phase is an oil phase and the continuous phase is an aqueous phase but interfacial polymerisation reactions at the interface of a continuous oil phase and a dispersed aqueous phase are also possible. Thus for example an oil or organic phase is dispersed into a continuous aqueous phase comprising water and a surface-active agent. The organic phase is dispersed as discrete droplets throughout the aqueous phase by means of emulsification, with an interface between the discrete organic phase droplets and the surrounding continuous aqueous phase solution being formed. Polymerisation at this interface forms the microcapsule shell surrounding the dispersed phase droplets.

In one type of interfacial condensation polymerisation microencapsulation process, monomers contained in the oil and aqueous phase respectively are brought together at the oil/water interface where they react by condensation to form the microcapsule wall. In another type of polymerisation reaction, the in situ interfacial condensation polymerisation reaction, all of the wall-forming monomers are contained in the oil phase. In situ condensation of the wall-forming materials and curing of the polymers at the organic-aqueous phase interface may be initiated by heating the emulsion to a temperature of between about 20° C. to about 100° C. and optionally adjusting the pH. The heating occurs for a sufficient period of time to allow substantial completion of in situ condensation of the prepolymers to convert the organic droplets to capsules consisting of solid permeable polymer shells enclosing the organic core materials.

One type of microcapsule prepared by in situ condensation and known in the art is exemplified in U.S. Pat. Nos. 4,956,129 and 5,332,584. These microcapsules, commonly termed “aminoplast” microcapsules, are prepared by the self-condensation and/or cross-linking of etherified urea-formaldehyde resins or prepolymers in which from about 50 to about 98% of the methylol groups have been etherified with a C₄-C₁₀ alcohol (preferably n-butanol). The prepolymer is added to or included in the organic phase of an oil/water emulsion. Self-condensation of the prepolymer takes place optionally under the action of heat at low pH. To form the microcapsules, the temperature of the two-phase emulsion is raised to a value of from about 20° C. to about 90° C., preferably from about 40° C. to about 90° C., most preferably from about 40° C. to about 60° C. Depending on the system, the pH value may be adjusted to an appropriate level. For the purpose of this invention a pH of about 1.5 to 3 is appropriate:

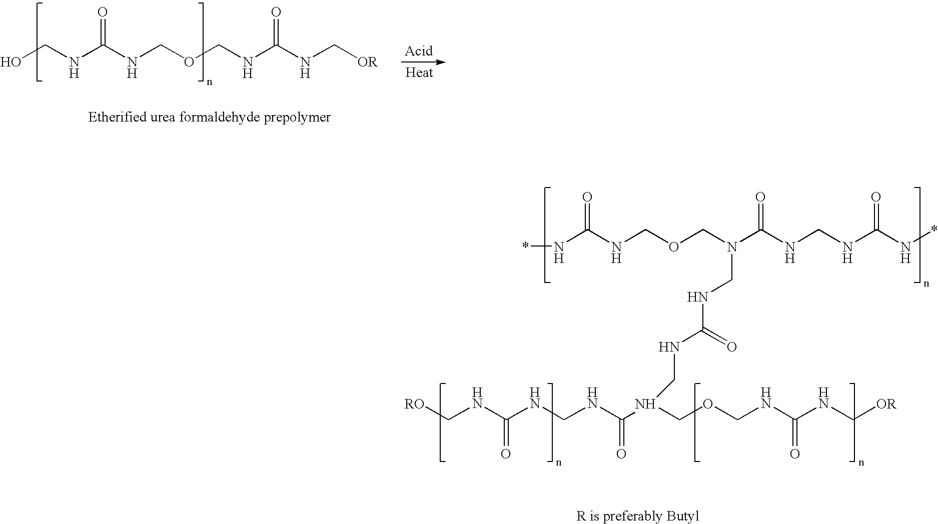

As described in U.S. Pat. No. 4,285,720 the prepolymers most suitable for use in this invention are partially etherified urea-formadehyde prepolymers with a high degree of solubility in organic phase and a low solubility in water. Etherified urea-formaldehyde prepolymers are commercially available in alcohol or in a mixture of alcohol and xylene.

Examples of preferred commercially available prepolymers include the Beetle etherified urea resins manufactured by BIP (e.g. BE607, BE610, BE660, BE676) or the Dynomin N-butylated urea resins from Dyno Cyanamid (e.g. Dynomin UB-24-BX, UB-90-BX etc.).

Acid catalysts capable of enhancing the microcapsule formation can be placed in either the aqueous or the organic phase. Catalysts are generally used when the core material is too hydrophobic, since they serve to attract protons towards the organic phase. Any water soluble catalyst which has a high affinity for the organic phase can be used. Carboxylic and sulphonic acids are particularly useful.

One further type of microcapsule prepared by in situ condensation and found in the art, as exemplified in U.S. Pat. No. 4,285,720 is a polyurea microcapsule which involves the use of at least one polyisocyanate such as polymethylene polyphenyleneisocyanate (PMPPI) and/or tolylene diisocyanate (TDI) as the wall-forming material. In the creation of polyurea microcapsules, the wall-forming reaction is generally initiated by heating the emulsion to an elevated temperature at which point a proportion of the isocyanate groups are hydrolyzed at the interface to form amines, which in turn react with unhydrolyzed isocyanate groups to form the polyurea microcapsule wall. During the hydrolysis of the isocyanate monomer, carbon dioxide is liberated. The addition of no other reactant is required once the dispersion establishing droplets of the organic phase within a continuous liquid phase, i.e., aqueous phase, has been accomplished. Thereafter, and preferably with moderate agitation of the dispersion, the formation of the polyurea microcapsule can be brought about by heating the continuous liquid phase or by introducing a catalyst such as an alkyl tin or a tertiary amine capable of increasing the rate of isocyanate hydrolysis.

The organic phase thus comprises the catalyst to be encapsulated, a polyisocyanate and optionally organic solvent. The catalyst can be in a concentrated form or as a solution in a water immiscible solvent. The catalyst to be encapsulated and the polyisocyanate are typically premixed under slow agitation to obtain a homogeneous organic phase before addition to and mixing with the aqueous phase. The amount of the organic phase may vary from about 1% to about 75% by volume of the aqueous phase present in the reaction vessel. The preferred amount of organic phase is about 10 percent to about 50 percent by volume. The organic polyisocyanates used in this process includes both aromatic and aliphatic mono and poly functional isocyanates. Examples of suitable aromatic diisocyantes and other polyisocyantes include the following: 1-chloro-2,4-phenylene diisocyante, m-phenylene diisocyante (and its hydrogenated derivative), p-phenylene diisocyante (and its hydrogenated derivative), 4,4′-methylenebis (phenyl isocyanate), 2,4-tolylene diisocyanate, tolylene diisocyanate (60% 2,4-isomer, 40% 2,6-isomer), 2,6-tolylene diisocyante, 3,3′-dimethyl-4,4′-biphenylene diisocyante, 4,4′-methylenebis (2-methylphenyl isocyanate), 3,3′-dimethoxy-4,4′-biphenylene diisocyanate, 2,2′,5,5′-tetramethyl-4,4′-biphenylene diisocyanate, 80% 2,4- and 20% 2,6-isomer of tolylene diisocyanate, polymethylene polyphenylisocyante (PMPPI), 1,6-hexamethylene diisocyanate, isophorone diisocyanate, tetramethylxylene diisocyanate and 1,5-naphthylene diisocyanate.

It may be desirable to use combinations of the above mentioned polyisocyantes. Preferred polyisocyantes are polymethylene polyphenylisocyante (PMPPI) and mixtures of polymethylene polyphenylisocyante (PMPPI) with tolylene diisocyanate.

One further class of polymer precursors consists of a primarily oil-soluble component and a primarily water-soluble component which react together to undergo interfacial polymerisation at a water/oil interface. Typical of such precursors are an oil-soluble isocyanate such as those listed above and a water-soluble poly amine such as ethylenediamine and/or diethylenetriamine to ensure that chain extension and/or cross-linking takes place. Cross-linking variation may be achieved by increasing the functionality of the amine. Thus for example, cross-linking is increased if ethylenediamine is replaced by a polyfunctional amine such as DETA (Diethylene triamine), TEPA (Tetraethylene pentamine) and other well established cross linking amines. Isocyanate functionality can be altered (and thus cross-linking also altered) by moving from monomeric isocyanates such as toluene diisocyanate to PMPPI. Mixtures of isocyanates, for example mixtures of tolylene diisocyanate and PMPPI, may also be used. Moreover, the chemistry may be varied from aromatic isocyanates to aliphatic isocyanates such as hexamethylenediisocyanate and isophorone diisocyanate. Further modifications can be achieved by partially reacting the (poly) isocyanate with a polyol to produce an amount of a polyurethane within the isocyanate chemistry to induce different properties to the wall chemistry. For example, suitable polyols could include simple low molecular weight aliphatic di, tri or tetraols or polymeric polyols. The polymeric polyols may be members of any class of polymeric polyols, for example: polyether, polyTHF, polycarbonates, polyesters and polyesteramides. One skilled in the art will be aware of many other chemistries available for the production of a polymeric wall about an emulsion droplet. As well as the established isocyanate/amine reaction to produce a polyurea wall chemistry, there can be employed improvements to this technology including for example that in which hydrolysis of the isocyanate is allowed to occur to an amine which can then further react internally to produce the polyurea chemistry (as described for example in U.S. Pat. No. 4,285,720). Variation in the degree of cross linking may be achieved by altering the ratio of monomeric isocyanate to polymeric isocyanate. As with the conventional isocyanate technology described above, any alternative isocyanates can be employed in this embodiment.

One skilled in the art will be aware that the various methods previously described to produce polyurea microcaps typically leave unreacted amine (normally aromatic amine) groups attached to the polymer matrix. In some cases it may be advantageous to convert such amine groups to a substantially inert functionality. Preferred are methods for the conversion of such amine groups to urea, amide or urethane groups by post reaction of the microcapsules in an organic solvent with a monoisocyanate, acid chloride or chloroformate respectively.

U.S. Pat. No. 6,020,066 (assigned to Bayer AG) discloses another process for forming microcapsules having walls of polyureas and polyiminoureas, wherein the walls are characterized in that they consist of reaction products of crosslinking agents containing NH₂ groups with isocyanates. The crosslinking agents necessary for wall formation include di- or polyamines, diols, polyols, polyfunctional amino alcohols, guanidine, guanidine salts, and compounds derived there from. These agents are capable of reacting with the isocyanate groups at the phase interface in order to form the wall.

The preferred materials for the microcapsule are a polyurea, formed as described in U.S. Pat. No. 4,285,720, or a urea-formaldehyde polymer as described in U.S. Pat. No. 4,956,129. Polyurea is preferred because the microcapsule is formed under very mild conditions and does not require acidic pH to promote polymerisation and so is suitable for use with an acid-sensitive catalysts. The most preferred polymer type for the microcapsule is polyurea as described in U.S. Pat. No. 4,285,720 based on the PMPPI polyisocyanate.

Whilst the scope of the present invention is not to be taken as being limited by any one particular theory, it is believed that certain microcapsule wall-forming moieties, such as for example isocyanate moieties, may provide co-ordinating functionality in respect to a transition metal catalyst. Such co-ordination may result in the possibility of stabilisation of finely dispersed or colloidal catalysts and/or the possibility of enhanced binding of the catalyst to the microcapsule polymer wall.

Certain organic or naturally occurring catalysts which act as ligands (for example tertiary amines) may interfere with reaction of the components forming the polymer microcapsule shell and it is preferred that the catalyst is an inorganic catalyst and in particular a transition metal catalyst. The term transition metal catalyst as used herein includes (a) the transition metal itself, normally in finely divided or colloidal form, (b) a complex of a transition metal with a suitable ligand or (c) a compound containing a transition metal. If desired a pre-cursor for the catalyst may be microencapsulated within the polymer microcapsule shell and subsequently converted to the catalyst, for example by heating. The term catalyst thus also includes a catalyst pre-cursor.

Microencapsulation techniques described above most commonly involve the microencapsulation of an oil phase dispersed within an aqueous continuous phase, and for such systems the catalyst is suitably capable of being suspended within the microencapsulated oil phase or more preferably is soluble in a water-immiscible organic solvent suitable for use as the dispersed phase in microencapsulation techniques. The scope of the present invention is not however restricted to the use of oil-in-water microencapsulation systems and water-soluble catalysts may be encapsulated via interfacial microencapsulation of water-in-oil emulsion systems. Water-soluble catalysts may also be encapsulated via interfacial microencapsulation of water-in-oil-in-water emulsion systems.

We have found that certain catalysts may catalyse the wall-forming reaction during interfacial polymerisation. In general it is possible to modify the microencapsulation conditions to take account of this. Some interaction, complexing or bonding between the catalyst and the polymer shell may be positively desirable since it may prevent agglomeration of finely divided or colloidal catalysts.

In some instances, the metal catalyst being encapsulated may increase the rate of the interfacial polymerisation reactions. In such cases it may be advantageous to cool one or both of the organic and continuous aqueous phases such that interfacial polymerisation is largely prevented whilst the organic phase is being dispersed. The reaction is then initiated by warming in a controlled manner once the required organic droplet size has been achieved. For example, in certain reactions the aqueous phase may be cooled to less than 10° C., typically to between 5° C. to 10° C., prior to addition of the oil phase and then when the organic phase is dispersed the aqueous phase may be heated to raise the temperature above 15° C. to initiate polymerisation.

Preferred transition metals on which the catalysts for use in the present invention may be based include platinum, palladium, osmium, ruthenium, rhodium, iridium, rhenium, scandium, cerium, samarium, yttrium, ytterbium, lutetium, cobalt, titanium, chromium, copper, iron, nickel, manganese, tin, mercury, silver, gold, zinc, vanadium, tungsten and molybdenum. Especially preferred transition metals on which the catalysts for use in the present invention may be based include palladium, osmium, ruthenium, rhodium, titanium, vanadium and chromium. Air sensitive catalysts may be handled using conventional techniques to exclude air.

An example of a water-soluble catalyst which may be encapsulated via a water-in oil emulsion microencapsulation process is scandium triflate.

Osmium in the form of osmium tetroxide is useful as a catalyst in a variety of oxidation reactions. Since it has a high vapour pressure even at room temperature and its vapour is toxic, microencapsulation of osmium tetroxide according to the present invention has the added advantage of a potential reduction in toxicity problems. Osmium tetroxide is soluble in solvents, in particular hydrocarbon solvents, which are suitable for forming the dispersed phase in a microencapsulation reaction.

Palladium in a variety of forms may be microencapsulated and is useful as a catalyst for a wide range of reactions according to the present invention. Colloidal palladium may be produced as an organic phase dispersion and is conveniently stabilised by quaternary ammonium salts such as tetra-n-octylammonium bromide. Thus for example colloidal palladium may be produced by the thermal decomposition of palladium acetate dissolved in a solvent such as tetrahydrofuran in the presence of tetra-n-octylammonium bromide as stabiliser. The tetrahydrofuran solvent is suitably removed, for example under reduced pressure, and may be replaced by a solvent which is water-immiscible and is hence more suitable for the microencapsulation process. Whilst such a colloidal suspension of palladium may be successfully microencapsulated, we have found that the stabilised palladium tends to catalyse the polymerisation reaction at the interface (probably via octylammonium bromide acting as a ligand) and it may be necessary to adjust the microencapsulation conditions accordingly.

Alternatively palladium may be used directly in the form of palladium acetate. Thus for example palladium acetate may be suspended or more preferably dissolved in a suitable solvent such as a hydrocarbon solvent or a chlorinated hydrocarbon solvent and the resultant solution may be microencapsulated to form a catalyst system for use in the present invention. Chloroform is a preferred solvent for use in the microencapsulation of palladium acetate. Whilst the scope of the present invention is not to be taken as being limited by any one particular theory, it is believed that the solubility of the catalyst in the organic phase is increased in the presence of an isocyanate microcapsule wall-forming moiety, either as a result of an increase in polarity of the organic phase or possibly via co-ordination with the metal.

According to literature sources palladium acetate decomposes to the metal under the action of heat. Catalysts systems derived from palladium acetate have proved to be effective in the process of the present invention, although it is not presently known whether palladium is present in the form of the metal or remains as palladium acetate.

It is to be understood that the microencapsulated catalysts for use in the process of the present invention include microencapsulated catalysts wherein the loading level of catalyst can be varied. Microencapsulated catalysts with loadings of 0.01 mmol/g to 0.6 mmol/g of catalyst are typical, especially where the loading is based on the metal content. Loadings of 0.2 mmol/g to 0.4 mmol/g are frequently favoured.

In addition to the metal catalysts and metal oxide catalysts, many additional catalysts which may be microencapsulated for use in the present invention will occur to those skilled in the art. Without limitation to the foregoing, the following are examples of suitable catalysts:—
Catalysts disclosed in Catalytic Asymmetric Synthesis 2nd Ed. Ed. I. Ojima Wiley-VCH including without limitation the list of chiral ligands included in the appendix thereof;
Metal diphosphine catalysts such as those disclosed in EP612758 Solvias RhJosiPhos, EP366390 Takasago RuBINAP, EP398132 Roche MeOBIPHEP, U.S. Pat. No. 5,008,457 DuPont DuPhos and PCT/GB99/03599 OxPhos;
Metal phosphine catalysts such as Wilkinson's catalysts disclosed in Chem. Rev., 1991, 91, 1179;
Metal phosphoramidate catalysts such as those disclosed in WO02/04466 DSM MonoPhos;
Metal aminophosphine catalysts such as those disclosed in A. Pfaltz Acc. Chem. Res. 1993, 26, 339, J. M. Brown, D. Hulmes, T. Layzell J. Chem. Soc. Chem. Commun. 22, 1673, 1993, and J. Am. Chem. Soc., 1992, 114, 9327;
Metal arylamine catalysts such as those disclosed in Organometallics, 1997, 16(23), 4985-4994;
Metal diamine catalysts such as those disclosed in U.S. Pat. No. 5,663,393 Jacobsen epoxidation, U.S. Pat. No. 5,637,739 Jacobsen epoxidation, U.S. Pat. No. 5,929,232 Jacobsen epoxide resolution, U.S. Pat. No. 4,871,855 Sharpless dihydroxylation, U.S. Pat. No. 5,260,461 Sharpless dihydroxylation, U.S. Pat. No. 5,767,304 Sharpless aminohydroxylation, U.S. Pat. No. 5,859,281 Sharpless aminohydroxylation, U.S. Pat. No. 6,008,376 Sharpless aminohydroxylation and WO02/10095 for Catalytic Asymmetric Cyanohydrin;
Metal aminoalcohol catalysts such as those disclosed in WO9842643 Zeneca CATHy, and EP0916637 ERATO Noyori CTH;
Metal phosphate catalysts such as those disclosed in Cserepi-Szucs, S., Bakos, J. Chem. Soc. Chem. Commun. 1997, 635;
Metal salt catalysts such as salts of magnesium, aluminium, tin and iron for instance halide salts such as chlorides of magnesium, aluminium, tin and iron;
Metal alkoxide catalysts such as those disclosed in Verdaguer X., Lange, U. E. W., Reding, M. T., Buchwald S. L. J. Am. Chem. Soc. 1996, 118, 6784;
Metal arene catalysts such as those disclosed in U.S. Pat. No. 5,489,682 Buchwald hydrogenation, U.S. Pat. No. 5,929,266 Whitby hydrogenation;
Metal arene phosphine catalysts such as those disclosed in Ciruelos, S., Englert, E., Salzer, A., Bolm, C., Maischak, A. Organometallics 19, 2240, 2000;
Metal carbene catalysts for alkene metathesis such as those described in J. Am. Chem. Soc., 1994, 116, 3414, J. Am. Chem. Soc., 1999, 121, 2674 and J. Am. Chem. Soc. 1993, 115, 9856; and
Metallocycle catalysts such as those described in Angew. Chem. 1995, 34, 1844 and Chem. Commun. 1998, 2095.

The microencapsulation of the catalyst takes place according to techniques well known in the art. Typically the catalyst is dissolved or dispersed in an oil phase which is emulsified into a continuous aqueous phase to form an emulsion which is generally stabilised by a suitable surfactant system. A wide variety of surfactants suitable for forming and stabilising such emulsions are commercially available and may be used either as the sole surfactant or in combination. The emulsion may be formed by conventional low or high-shear mixers or homogenisation systems, depending on particle size requirements. A wide range of continuous mixing techniques can also be utilised. Suitable mixers which may be employed in particular include dynamic mixers whose mixing elements contain movable parts and static mixers which utilise mixing elements without moving parts in the interior. Combinations of mixers (typically in series) may be advantageous. Examples of the types of mixer which may be employed are discussed in U.S. Pat. No. 627,132 which is herein incorporated by reference. Alternatively, emulsions may be formed by membrane emulsification methods. Examples of membrane emulsification methods are reviewed in Journal of Membrane Science 169 (2000) 107-117 which is herein incorporated by reference.

Typical examples of suitable surfactants include:
a) condensates of alkyl (eg octyl, nonyl or polyaryl) phenols with ethylene oxide and optionally propylene oxide and anionic derivatives thereof such as the corresponding ether sulphates, ether carboxylates and phosphate esters; block copolymers of polyethylene oxide and polypropylene oxide such as the series of surfactants commercially available under the trademark PLURONIC (PLURONIC is a trademark of BASF);
b) TWEEN surfactants, a series of emulsifiers comprising a range of sorbitan esters condensed with various molar proportions of ethylene oxide;
c) condensates of C₈ to C₃₀ alkanols with from 2 to 80 molar proportions of ethylene oxide and optionally propylene oxide; and
d) polyvinyl alcohols, including the carboxylated and sulphonated products.

Furthermore, WO 01/94001 teaches that one or more wall modifying compounds (termed surface modifying agents) can, by virtue of reaction with the wall forming materials, be incorporated into the microcapsule wall to create a modified microcapsule surface with built in surfactant and/or colloid stabiliser properties. Use of such modifying compounds may enable the organic phase wall forming material to be more readily dispersed into the aqueous phase possibly without the use of additional colloid stabilisers or surfactants and/or with reduced agitation. The teaching of WO01/94001 is herein incorporated by reference. Examples of wall modifying compounds which may find particular use in the present invention include anionic groups such as sulphonate or carboxylate, non-ionic groups such as polyethylene oxide or cationic groups such as quaternary ammonium salts.

In addition the aqueous phase may contain other additives which may act as aids to the process of dispersion or the reaction process. For example, de-foamers may be added to lesson foam build up, especially foaming due to gas evolution.

A wide variety of materials suitable for use as the oil phase will occur to one skilled in the art. Examples include, diesel oil, isoparaffin, aromatic solvents, particularly alkyl substituted benzenes such as xylene or propyl benzene fractions, and mixed napthalene and alkyl napthalene fractions; mineral oils, white oil, castor oil, sunflower oil, kerosene, dialkyl amides of fatty acids, particularly the dimethyl amides of fatty acids such as caprylic acid; chlorinated aliphatic and aromatic hydrocarbons such as 1,1,1-trichloroethane and chlorobenzene, esters of glycol derivatives, such as the acetate of the n-butyl, ethyl, or methyl ether of diethylene glycol, the acetate of the methyl ether of dipropylene glycol, ketones such as isophorone and trimethylcyclohexanone (dihydroisophorone) and the acetate products such as hexyl, or heptyl acetate. Organic liquids conventionally preferred for use in microencapsulation processes are xylene, diesel oil, isoparaffins and alkyl substituted benzenes, although some variation in the solvent may be desirable to achieve sufficient solubility of the catalyst in the oil phase.

It is preferred that microencapsulation of the oil phase droplets containing the catalyst takes place by an interfacial polymerisation reaction as described above. The aqueous dispersion of microcapsules containing the catalyst may be used to catalyse a suitable reaction without further treatment. Preferably however the microcapsules containing the catalyst are removed from the aqueous phase by filtration. It is especially preferred that the recovered microcapsules are washed with water to remove any remaining surfactant system and with a solvent capable of extracting the organic phase contained within the microcapsule. Relatively volatile solvents such as halogenated hydrocarbon solvents for example chloroform are generally more readily removed by washing or under reduced pressure than are conventional microencapsulation solvents such as alky substituted benzenes. If the majority of the solvent is removed, the resultant microcapsule may in effect be a substantially solvent-free polymer bead containing the catalyst efficiently dispersed within the microcapsule polymer shell. The process of extracting the organic phase may cause the microcapsule walls to collapse inward, although the generally spherical shape will be retained. If desired the dry microcapsules may be screened to remove fines, for example particles having a diameter less than about 20 microns.

In the case of the microencapsulated palladium acetate microparticles it is preferred that the recovered water wet microcapsules are washed with copious quantities of deionised water, followed by ethanol washes and finally hexane washes. The microcapsules are then dried in a vac oven at 50° C. for approx 4 hours to give a product with greater than 98% non volatile content (by exhaustive drying).

Depending on the conditions of preparation and in particular the degree of interaction between the catalyst and the wall-forming materials, the microencapsulated catalyst of the present invention may be regarded at one extreme as a ‘reservoir’ in which the finely divided catalyst (either as solid or in the presence of residual solvent) is contained within an inner cavity bound by an integral outer polymer shell or at the other extreme as a solid, amorphous polymeric bead throughout which the finely divided catalyst is distributed. In practice the position is likely to be between the two extremes. Regardless of the physical form of the encapsulated catalyst of the present invention and regardless of the exact mechanism by which access of reactants to the catalyst takes place (diffusion through a permeable polymer shell or absorption into a porous polymeric bead), we have found that encapsulated catalysts of the present invention permit effective access of the reactants to the catalyst whilst presenting the catalyst in a form in which it can be recovered and if desired re-used. Furthermore, since in the preferred embodiment of the present invention the polymer shell/bead is formed in situ by controlled interfacial polymerisation (as opposed to uncontrolled deposition from an organic solution of the polymer), the microencapsulated catalyst of the present invention may be used in a wide range of organic solvent-based reactions.

The microcapsules for use in the process of this invention are regarded as being insoluble in most common organic solvents by virtue of the fact that they are highly crosslinked. As a consequence, the microcapsules can be used in a wide range of organic solvent based reactions.

The microcapsules containing the catalyst may be added to the reaction system to be catalysed and, following completion of the reaction, may be recovered for example by filtration. The recovered microcapsules may be returned to catalyse a further reaction and re-cycled as desired. Alternatively, the microcapsules containing the catalyst may be used as a stationary catalyst in a continuous reaction. For instance, the microcapsule particles could be immobilised with a porous support matrix (e.g. membrane). The microcapsule is permeable to the extent that catalysis may take place either by diffusion of the reaction medium through the polymer shell walls or by absorption of the reaction medium through the pore structure of the microcapsule.

It will be appreciated that the use of microencapsulated catalysts under supercritical or near supercritical conditions may be used for any reaction appropriate to that catalyst and that the scope of the present invention is not limited to use of the catalyst in any particular reaction type or reaction medium. Examples of the types of reactions in which it may be appropriate to use a microencapsulated catalyst under supercritical or near supercritical conditions include Suzuki couplings, Heck reactions, Stille reactions, hydrogenations, allylic alkylations, Sharpless asymmetric dihydroxylation and reactions which are generally known which utilise palladium acetate as a catalyst, for instance, those reactions discussed in Palladium Reagents and Catalysts, Tsuji, J., Published by Wiley (Chichester) 1995; Metal Catalysed Cross-Coupling Reactions, Edited by Diederich, F., and Stang P. J., Published by Wiley-VCH (Weinham) 1998; Comprehensive Organometallic Chemistry, 2nd Ed., Farina V., Edited by Abel E. W., Stone F. G., and Wilkinson G., Published by Pergamon (London) 1995; Vol 12, p161; and Transition Metal Reagents and Catalysis, Tsuji J., Published by Wiley (Chichester) 2000.

The term “supercritical or near supercritical conditions” includes those conditions of temperature, pressure under which certain solvent mediums are known to form a supercritical or a near supercritical fluid.

A fluid is termed supercritical when its temperature exceeds the critical temperature (Tc). At this point the two fluid phases, liquid and vapour, become indistinguishable see A. Baiker, Chem. Rev, 1999, 99, 453-474 (section III p. 455).

Conditions and solvent mediums required to form supercritical or near supercritical states are described in Oakes, R. Scott, Clifford, Anthony A., and Rayner, Christopher M., Journal of the Chemical Society, Perkin Transactions 1 2001, 9, 917-941; Shezad, N., Oakes, R. S., Clifford, A. A., and Rayner, C. M., Chemical Industries (Dekker) 2001, 82 (Catalysis of Organic Reactions), 459-464; Shezad, Najam, Clifford, Anthony A., and Rayner, Christopher M. Green Chemistry 2002, 4(1), 64-67; and in WO96013404; WO9522591; WO9420444; WO9406738; EP0652202; and U.S. Pat. No. 6,156,933 which are herein incorporated by reference.

The term “supercritical or near supercritical conditions” also includes conditions of temperature, pressure under which certain solvent mediums are often referred to as compressed mediums. This includes compressed mediums such as compressed ethane, compressed propane, and especially compressed CO₂.

In general many of the reactions take place in solvent mediums or mixtures of solvent mediums which are chosen for their ability to form supercritical or near supercritical fluids. Additionally, certain solvent mediums may cause the microcapsule polymer to swell and this may aid the contact of the reactants with the catalyst.

Any solvent medium which is capable of forming a supercritical or near supercritical fluid can be employed. Solvent mediums capable of forming a supercritical or near supercritical fluid include low molecular weight hydrocarbons, particularly C₂₋₄alkanes, freons, ethers, particularly dimethyl ether, carbon dioxide, ammonia, water, nitrous oxide and mixtures thereof. Preferred solvent mediums include low molecular weight hydrocarbons, particularly C₂₋₄alkanes, freons, carbon dioxide and mixtures thereof. Most preferred solvent medium is carbon dioxide.

Examples of solvent mediums include ethane, propane, butane, CO₂, dimethyl ether, N₂O, water, and ammonia.

It is preferred that the solvent medium is chosen such that both that the substrate and products of the reaction form a substantially homogenous mixture with the solvent medium and that this homogenous mixture is in a supercritical or near supercritical state.

Although processes according to the present invention can be carried out under any conditions of temperature, pressure and in any solvent medium known to form a supercritical or near supercritical state. Typically the substrate of the reaction will initially be present in a concentration which is in part dependent on the solvent medium. Certain reactions may favour relatively low concentrations of substrate being employed. Typically the substrate and solvent medium would be brought to a super-critical or near super-critical state at a temperature between 45 and 274° C. according to the actual fluid selected.

Examples of the critical temperature (Tc) and pressures (Pc) of solvent mediums which may be employed in the processes of the present invention are shown below:

| Solvent | T_{c}(° C.) | P_{c} (MPa) |
|---|---|---|
| ethane | 32.3 | 4.88 |
| propane | 96.7 | 4.25 |
| butane | 152.1 | 3.80 |
| hexane | 234.1 | 2.97 |
| ethane | 9.2 | 5.04 |
| propene | 91.9 | 4.62 |
| 1-butene | 146.5 | 3.97 |
| 2-trans-butene | 155.5 | 3.99 |
| dimethyl ether | 126.9 | 5.24 |
| tetrafluoromethane | −45.6 | 3.74 |
| hexafluoroethane | 19.7 | 2.98 |
| octafluoropropane | 71.9 | 2.68 |
| hexafluoropropylene | 94.0 | 2.90 |
| difluoromethane | 78.5 | 5.34 |
| trifluoromethane | 26.2 | 4.86 |
| chlorotrifluoromethane | 28.8 | 3.87 |
| chlorodifluoromethane | 96.2 | 4.97 |
| difluoroethane | 113.1 | 4.52 |
| tetrafluoroethane | 101.1 | 4.06 |
| pentafluoroethane | 66.3 | 3.63 |
| sulfur hexafluoride | 45.4 | 3.76 |
| carbon dioxide | 31.0 | 7.38 |
| nitrous oxide | 36.4 | 7.255 |
| water | 373.9 | 22.06 |
| ammonia | 132.3 | 11.35 |
| methanol | 239.4 | 8.092 |
| 1-propanol | 263.6 | 5.170 |
| 2-propanol | 235.1 | 4.762 |
| xenon | 16.5 | 5.84 |

Particularly preferred is the use of microencapsulated catalysts under supercritical or near supercritical conditions for metal mediated cross coupling reactions, most preferably Heck, Stille and Suzuki reactions, and for hydrogenation reactions, particularly transfer hydrogenation reactions.

According to a further aspect of the present invention there is provided a process for the preparation of optionally substituted biphenyls which comprises reacting an optionally substituted aryl halide or halide equivalent with an optionally substituted aryl boronic acid or ester in the presence of a catalyst system comprising a catalyst microencapsulated within a permeable polymer microcapsule shell under supercritical or near supercritical conditions.

According to a further aspect of the present invention there is provided a process for the preparation of optionally substituted biphenyls which comprises reacting an optionally substituted aryl halide or halide equivalent with a tri-alkylaryltin in the presence of a catalyst system comprising a catalyst microencapsulated within a permeable polymer microcapsule shell under supercritical or near supercritical conditions.

Preferred catalyst systems for use in the above two processes are as described hereinbefore. Preferably, the microcapsule shell is formed by interfacial polymerisation. More preferably the catalyst is based on palladium, colloidal palladium or palladium acetate being most preferred.

The optionally substituted aryl halide includes an optionally substituted aryl iodides, bromides or chlorides. Optionally substituted aryl halide equivalents include optionally substituted aryl compounds having an OTf substituent (where Tf=SO₂CF₃).

Preferred processes include the following: 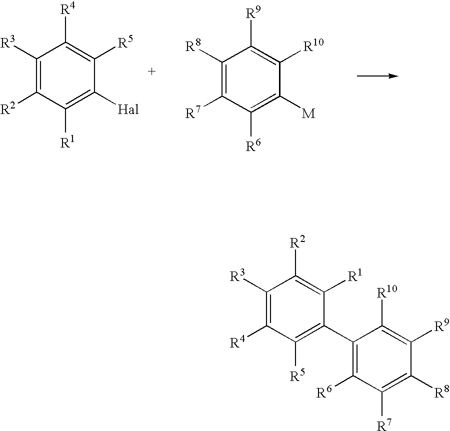
wherein:
Hal is a halide, preferably chloride, bromide or iodide, or a halide equivalent, preferably OTf;
R¹ to R¹⁰ are each independently hydrogen or a substituent group; and
M is B(OH)₂, B(OR¹¹)₂ or Sn(R¹²)₃ wherein R¹¹ is an alkyl or aryl group; and
R¹² is an alkyl group.
   When any of R¹ to R¹⁰ are a substituent group, the group should be selected so as not to adversely affect the rate or selectivity of the reaction. Substituent groups include F, CN, NO₂, OH, NH₂, SH, CHO, CO₂H, acyl, hydrocarbyl, perhalogenated hydrocarbyl, heterocyclyl, hydrocarbyloxy, mono or di-hydrocarbylamino, hydrocarbylthio, esters, carbonates, amides, sulphonyl, sulphonamido and sulphonic acid ester groups wherein the hydrocarbyl groups include alkyl, and aryl groups, and any combination thereof, such as aralkyl and alkaryl, for example benzyl groups.

Alkyl groups which may be represented by R¹⁻¹⁰ include linear and branched alkyl groups comprising up to 20 carbon atoms, particularly from 1 to 7 carbon atoms and preferably from 1 to 5 carbon atoms. When the alkyl groups are branched, the groups often comprising up to 10 branch chain carbon atoms, preferably up to 4 branch chain atoms. In certain embodiments, the alkyl group may be cyclic, commonly comprising from 3 to 10 carbon atoms in the largest ring and optionally featuring one or more bridging rings. Examples of alkyl groups which may be represented by R¹⁻¹⁰ include methyl, ethyl, propyl, 2-propyl, butyl, 2-butyl, t-butyl and cyclohexyl groups.

Aryl groups which may be represented by R¹⁻¹⁰ may contain 1 ring or 2 or more fused rings which may include cycloalkyl, aryl or heterocyclic rings. Examples of aryl groups which may be represented by R¹⁻¹⁰ include phenyl, tolyl, fluorophenyl, chlorophenyl, bromophenyl, trifluoromethylphenyl, anisyl, naphthyl and ferrocenyl groups.

Perhalogenated hydrocarbyl groups which may be represented by R¹-R¹⁰ independently include perhalogenated alkyl and aryl groups, and any combination thereof, such as aralkyl and alkaryl groups. Examples of perhalogenated alkyl groups which may be represented by R¹⁻¹⁰ include —CF₃ and —C₂F₅.

Heterocyclic groups which may be represented by R¹⁻¹⁰ independently include aromatic, saturated and partially unsaturated ring systems and may constitute 1 ring or 2 or more fused rings which may include cycloalkyl, aryl or heterocyclic rings. The heterocyclic group will contain at least one heterocyclic ring, the largest of which will commonly comprise from 3 to 7 ring atoms in which at least one atom is carbon and at least one atom is any of N, O, S or P. Examples of heterocyclic groups which may be represented by R¹⁻¹⁰ include pyridyl, pyrimidyl, pyrrolyl, thiophenyl, furanyl, indolyl, quinolyl, isoquinolyl, imidazoyl and triazoyl groups.

Preferably one or more of R¹, R⁵, R⁶ or R¹⁰ is hydrogen. Most preferably at least three of R¹, R⁵, R⁶ or R¹⁰ are hydrogen.

The processes may advantageously be used in the production of biphenyls where one or more of R², R⁴, R⁷ or R⁹ are a cyano group.

According to a further aspect of the present invention there is provided a process for the preparation of optionally substituted alkenes which comprises reacting an optionally substituted aryl halide or halide equivalent with an alkene optionally substituted with up to three substituents in the presence of a catalyst system comprising a catalyst microencapsulated within a permeable polymer microcapsule shell under supercritical or near supercritical conditions.

Preferred catalyst systems for use in the above two processes are as described hereinbefore. Preferably, the microcapsule shell is formed by interfacial polymerisation. More preferably the catalyst is based on palladium, colloidal palladium or palladium acetate being most preferred.

The optionally substituted aryl halide includes an optionally substituted aryl iodides, bromides or chlorides. Optionally substituted aryl halide equivalents include optionally substituted aryl compounds having an OTf substituent (where Tf=SO₂CF₃).

Preferred processes include the following: 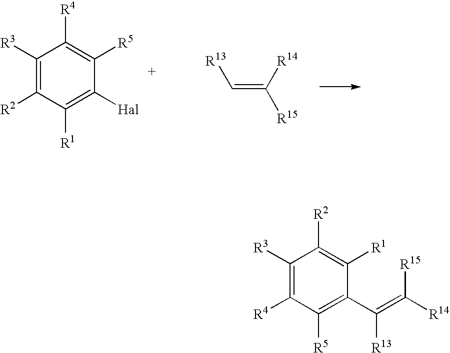
wherein:
Hal is a halide, preferably chloride, bromide or iodide, or a halide equivalent, preferably OTf;
R¹ to R⁵ are each independently hydrogen or a substituent group; and
R¹³ to R¹⁵ are each independently hydrogen or a substituent group.
   When any of R¹ to R⁵ are a substituent group, the group should be selected so as not to adversely affect the rate or selectivity of the reaction. Substituent groups include F, CN, NO₂, OH, NH₂, SH, CHO, CO₂H, acyl, hydrocarbyl, perhalogenated hydrocarbyl, heterocyclyl, hydrocarbyloxy, mono or di-hydrocarbylamino, hydrocarbylthio, esters, carbonates, amides, sulphonyl, sulphonamido and sulphonic acid ester groups wherein the hydrocarbyl groups include alkyl, and aryl groups, and any combination thereof, such as aralkyl and alkaryl, for example benzyl groups.

R¹³ to R¹⁵ are preferably selected from the substituent groups listed above for R¹. Optionally one or more of R¹³ & R¹⁴ or R¹⁴ & R¹⁵ may be joined to form an optionally substituted ring. When any of R¹³ & R¹⁴ or R¹⁴ & R¹⁵ are joined to form an optionally substituted ring, the ring may optionally form part of a fused ring system. Preferably, when any of R¹³ & R¹⁴ or R¹⁴ & R¹⁵ are joined to form an optionally substituted ring, the ring preferably contains 5, 6 or 7 ring atoms which are preferably carbon atoms.

Most preferably, one or more of R¹³ to R¹⁵ are selected from CN, NO₂, acyl, ester hydrocarbyl, and hydrocarbyloxy groups.

Surprisingly the encapsulated palladium reagent affords superior yields in the Heck and Suzuki cross coupling reactions in sc CO₂ involving the usually unreactive aryl bromides and aryl chlorides. Furthermore the use of this catalyst affords superior yields of such cross couplings in sc CO₂ than are obtained in conventional solvents.

In another aspect of the invention the encapsulated palladium catalyst surprisingly promotes efficient Heck and Suzuki reactions in sc CO₂ in the absence of the conventional organophosphine ligands which are required in traditional solvents.

Low catalyst loadings are a feature of the present invention. Whereas typical loadings of unsupported palladium(II) acetate are normally in the range of 1 mol % to achieve reasonable conversions the encapsulated catalyst can be used in levels as low as 0.04 mol %.

A surprising aspect of the process using the encapsulated palladium is that tetra-alkylammonium salts are suitable co-additives for the Heck and Suzuki reactions of aryl bromides and aryl chlorides in sc CO₂, and thus promote unexpectedly high yields of coupled products. Tetraalkylammonium bromide, chloride and acetate salts have been used as molten solvents for Heck reactions (V. P. W. Böhm and W. A. Herrmann, Chem. Eur. J., 2000, 6, 1017). The above described combination in sc CO₂ is surprisingly superior.

According to a further aspect of the present invention there is provided a process for the preparation of diols which comprises reacting an olefin in the presence of a catalyst system comprising osmium tetroxide microencapsulated within a permeable polymer microcapsule shell under supercritical or near supercritical conditions.

Preferred processes include the following: 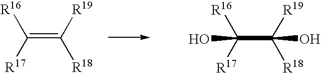
wherein:
R¹⁶ to R¹⁹ are each independently hydrogen or a substituent group.

Most preferably two or more of R¹⁵ to R¹⁹ are substituent groups.

When any of R¹⁶ to R¹⁹ are a substituent group, the group should be selected so as not to adversely affect the rate or selectivity of the reaction. Substituent groups include halide, CN, NO₂, OH, NH₂, SH, CHO, CO₂H, acyl, hydrocarbyl, perhalogenated hydrocarbyl, heterocyclyl, hydrocarbyloxy, mono or di-hydrocarbylamino, hydrocarbylthio, esters, carbonates, amides, sulphonyl, sulphonamido and sulphonic acid ester groups wherein the hydrocarbyl groups include alkyl, and aryl groups, and any combination thereof, such as aralkyl and alkaryl, for example benzyl groups.

Optionally one or more of R¹⁶ & R¹⁷, R¹⁷ & R¹⁸, R¹⁸ & R¹⁹ or R¹⁶ & R¹⁹ may be joined to form an optionally substituted ring. When any of R¹⁶ & R¹⁷, R¹⁷ & R¹⁸, R¹⁸ & R¹⁹ or R¹⁶ & R¹⁹ are joined to form an optionally substituted ring, the ring may optionally form part of a fused ring system. Preferably, when any of R¹⁶ &R¹⁷, R¹⁷& R¹⁸, R¹⁸ & R¹⁹ or R¹⁶ & R¹⁹ are joined to form an optionally substituted ring, the ring preferably contains 5, 6 or 7 ring atoms which are preferably carbon atoms.

According to a further aspect of the present invention there is provided a process for preparation of a hydrogenated product comprising reacting a substrate, wherein the substrate contains a hydrogenatable group or bond, with hydrogen in the presence of a catalyst system comprising a catalyst microencapsulated within a permeable polymer microcapsule shell under supercritical or near supercritical conditions.

Preferred catalyst systems for use in the above two processes are as described hereinbefore. Preferably, the microcapsule shell is formed by interfacial polymerisation. More preferably the catalyst is based on palladium, colloidal palladium or palladium acetate being most preferred.

Substrates which contain a hydrogenatable group or bond include organic compounds with carbon-carbon double or triple bonds, particularly optionally substituted alkenes or alkynes, and organic compounds substituted with groups such as nitro, nitroso, azido and other groups which are susceptible to reduction by hydrogen in the presence of a metal catalyst.

Advantageously, selective reduction of one type of hydrogenatable group or bond in the presence of other types of groups or bonds which are susceptible to reduction by hydrogen may be achieved by use of the catalyst systems of the present invention under appropriate conditions.

In a further aspect of this invention it has been found that the encapsulated palladium catalyst can be employed for metal-catalysed cross coupling reactions under a continuous flow system which represents a manufacturing process. Encapsulated transition metal catalysts are promising solid phase supports for a range of transition metal mediated C—C bond forming processes and related carbometallation reactions in sc CO₂. Surprisingly, delivery of the reactants and CO₂ solvent by pumping through separate nozzles, followed by mixing in a reactor tube leads to extremely rapid chemical reaction under conditions above the critical temperature and pressure. The products and starting material emerge from the pressure reactor through a filter and control of back pressure determines the rate of product release. This procedure is applied to Suzuki, Heck, Sonogashira and Stille reactions. Most preferably the Suzuki reaction can be carried out under continuous flow conditions. Rapid formation of product is observed even when reactants are in contact through a single passage through the chamber involving a short residence time in contact with the catalyst. Optimally the flow conditions employ cosolvent loading of some reactants. The preferred cosolvents are methanol and toluene, but any selection of common solvents including fluorinated solvents may be used. Preferred Suzuki reaction is the cross coupling of phenylboronic acid with bromobenzene. The surprising success of Suzuki coupling of bromobenzene under conditions of such short contact times of reactants with the catalyst is noteworthy.

The invention is illustrated by the following examples.

### EXAMPLE 1

This Example illustrates the encapsulation of Pd(OAc)₂ in a polyurea matrix. Pd(OAc)₂ (0.4 g Aldrich, 98%) was suspended in Solvesso 200 (5 g) and the solution stirred for 20 min. To this mixture, polymethylene polyphenylene di-isocyanate (PMPPI) (4 g) was added and stirred for a further 20 min. The mixture was then added to an aqueous mixture containing REAX 100 M (1.8 g), TERGITOL XD (0.3 g) and Poly Vinyl Alcohol (PVOH) (0.6 g) in deionised water (45 ml) while shearing (using a FISHER rotary flow impeller) at 1000 rpm for 1 minute. The micro-emulsion thus obtained was paddle-stirred at room temperature for 24 h. The microcapsules obtained were filtered though a polyethylene frit (20 micron porosity) and the capsules were washed in the following order: deionised water (10×50 ml), ethanol (10×50 ml), acetone (10×50 ml), dichloromethane (2×10 ml), hexane (3×50 ml), ether (1×50 ml), and dried. Typical loading of Pd(OAc)₂ in microcapsules was 0.12 mmol/g (based on Pd analysis).

### EXAMPLE 2

This Example illustrates an alternative procedure for encapsulation of Pd(OAc)₂ in a polyurea matrix.

A mixture of Pd(OAc)₂ (5 g) and polylmethylene polyphenylene di-isocyanate (PMPPI, 50 g) in dichloroethane (70 mL) was stirred for 1 h at room temperature. The resulting dark solution was added at a steady rate to an aqueous mixture containing REAX 100 M (10 g), TERGITOL XD (2.5 g) and GOSHENOL (5 g) in de-ionised water (250 mL) while shearing (using a HEIDOLPH radial flow impeller, 50 mm) at 800 rpm for 2 minutes. The resulting oil-in-water emulsion was paddle-stirred (or shaker-stirred) at room temperature for 16 hours. Ethylene diamine (5 g) was added and the mixture paddle-stirred (or shaker-stirred) for 6 hours. The polyurea microcapsules obtained were filtered though a polyethylene frit (20-micron porosity) and were washed with de-ionised water, acetone, ethanol, ether and dried.

### EXAMPLE 3

This Example illustrates the encapsulation of colloidal palladium nanoparticles in a polyurea matrix.

Step 1: Preparation of Colloidal Palladium

Pd(OAc)₂ (0.3 g, Aldrich 98%) and tetra-n-octylammonium bromide (1.46 g, 3 equiv., Aldrich 98%) were dissolved in dry tetrahydrofuran (250 ml) and refluxed for 5 hours under argon. The solvent was removed under reduced pressure to a volume of about 50 ml, and 20 g of SOLVESSO 200 was added and the excess tetrahydrofuran removed under reduced pressure.

Step 2: Encapsulation of Colloidal Palladium

PMPPI (9 g) was added to the above solution of Solvesso 200 containing colloidal palladium. The mixture was quickly added to an aqueous mixture containing REAX 100 M (1.8 g), TERGITOL XD (0.3 g) and PVOH (0.6 g) in deionised water (45 ml) while shearing (using a Fisher rotary flow impeller) at 1000 rpm for 1 minute. The microemulsion thus obtained was paddle stirred at room temperature for 24 hours. The microcapsules were filtered though a polyethylene frit (20 micron porosity) and the capsules were washed in the following order: deionised water (10×50 ml), ethanol (10×50 ml), acetone (10×50 ml), dichloromethane (2×10 ml), hexane (3×50 ml), ether (1×50 ml), and dried.

### EXAMPLE 4

This Example illustrates the encapsulation of osmium tetroxide in a polyurea matrix.

PMPPI (3 g) was added to a solution of SOLVESSO 200 (3 g) containing osmium tetroxide (0.132 g). The resulting dark solution was added at a steady rate to an aqueous mixture containing REAX 100 M (0.6 g), TERGITOL XD (0.1 g) and polyvinyl alcohol (PVA) (0.2 g) in deionised water (15 ml) while shearing (using a Heidolph radial flow impeller, 30 mm) at 750 rpm for 1 minute. The resulting oil-in-water emulsion was paddle stirred (100 rpm) at room temperature for 48 hours. The polyurea microcapsules obtained were filtered though a polyethylene frit (20 micron porosity) and the capsules were washed in the following order: deionised water (10×50 ml), ethanol (10×50 ml), acetone (10×50 ml), hexane (3×50 ml), ether (1×50 ml) and dried.

### EXAMPLE 5

Encapsulated Pd was added to the reaction chamber on top of the filter, before the reactor was pressurised. Once reaction conditions (100° C. and 140 bar) had been reached, the reagents (phenylboronic acid, bromobenzene and tetrabutylammonium acetate) were added in a solution of methanol. Rapid conversions to biphenyl were seen for these reactions. The contact time for reagents meeting these solid catalysts in a continuous flow set up is very short. Multipass conditions are available for increasing yields. The concept of catalyst recycling was proved to be possible with the recycling of the encapsulated Pd catalyst three times (Table 1). The recycled runs not only show no significant drop off in conversion but in fact proceed at higher conversions than the initial run.

TABLE 1
Continuous flow Suzuki reaction in sc CO₂

| Entry | Run | Yield (%) |
|---|---|---|
| 1 | initial | 9 |
| 2 | recycle 1 | 16 |
| 3 | recycle 2 | 16 |

### EXAMPLE 6

### Heck Reactions

The following results and experimental show the surprising results for Heck reactions in sc CO₂. It is noteworthy that tetraalklyammonium salts, tetraalkyammonium acetate in particular, are important elements. The inventive aspect of these results is that this biphasic system is able to facilitate successful cross-coupling reaction with the encapsulated Pd catalyst in the absence of any phosphine or fluorine. Provisional experiments on the recycling ability of the catalyst are promising.

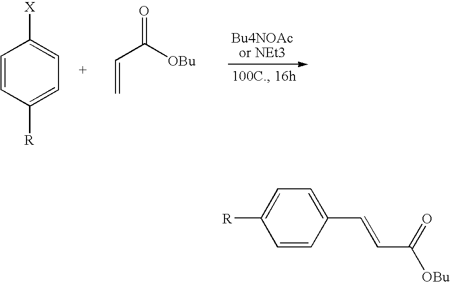

TABLE 2
Heck reactions with encapsulated Pd catalyst

| Entry | X | R | Base | Mol % Pd | Yielda |
|---|---|---|---|---|---|
| 1 | Br | NO₂ | Bu₄NOAc | 4 × 10⁻³ | 93 (95)^{b} |
| 2 | Br | NO₂ | NEt₃ | 1 × 10⁻¹ | 100 |
| 3 | Cl | NO₂ | Bu₄NOAc | 4 × 10⁻³ | 58^{c} |
| 4 | Br | H | Bu₄NOAc | 4 × 10⁻³ | 99 |
| 5 | Br | F | Bu₄NOAc | 4 × 10⁻³ | 75^{c} |
| 6 | Br | OMe | Bu₄NOAc | 4 × 10⁻³ | 99 |

Typical experimental conditions: aryl halide (1 mmol), tolylboronic acid (2 mmol), palladium catalyst (2.5 mmol) and tetrabutylammonium acetate (2-3 mmol);
^{a}Isolated yield;
^{b}Unoptimised

### EXAMPLE 7

### n-Butyl Cinnamate

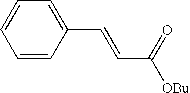

a) To a stainless steel reactor (10 ml) was added bromobenzene (0.1 ml, 0.9 mmol), butyl acrylate (0.2 ml, 1.3 mmol), encapsulated Pd resin as prepared in Example 1 (10 mg, 2.5 mmol) and tetrabutylammonium acetate (600 mg, ca 2 mmol). This was sealed under an atmosphere of CO₂ (ca. 800 psi). The reaction was heated at 100° C. for 3 h, upon which the reaction was cooled to room temperature and vented into a beaker containing EtOAc (50 ml). HPLC analysis of the mixture yield a modest yield of n-butyl cinnamate (5%). Heating an identical reaction for 16 h afforded n-butyl cinnamate in 47% yield (isolated).

b) Repeating the above procedure using 20 mg of encapsulated Pd (5 mmol) gave n-butyl cinnamate in 64% yield (115 mg).

c) Repeating the procedure in (a) with stirring, afforded n-butyl cinnamate in quantitative yield after chromatography (Ethyl acetate-hexane 1:99 as eluent).

### EXAMPLE 8

### n-Butyl-4-nitrocinnamate

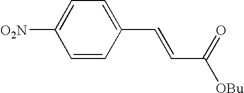

a) To a stainless steel reactor (10 ml) was added 4-bromonitrobenzene (190 mg, 0.95 mmol), butyl acrylate (0.2 ml, 1.3 mmol), encapsulated Pd resin as prepared in Example 1 (10 mg, 2.5 mmol) and tetrabutylammonium acetate (600 mg, ca 2 mmol). This was sealed under an atmosphere of CO₂ (ca. 800 psi). The reaction was heated at 100° C. for 16 h, upon which the reaction was cooled to room temperature and vented into a beaker containing EtOAc (50 ml). The cell was rinsed with ethyl acetate and the washings pooled with the vented solution. Silica gel was added (ca 10 g) and the solvent was removed under reduced pressure. Column chromatography of the product (loaded with adsorbed silica, ethyl acetate/hexane 1:4 as eluent) afforded n-butyl-4-nitrocinnamate in 96%.

b) To a stainless steel reactor (10 ml) was added 4-bromonitrobenzene (400 mg, 2 mmol), butyl acrylate (0.4 ml, 2.6 mmol), encapsulated Pd resin as prepared in Example 1 (10 mg, 0.2 mmol) and triethylamine (0.4 ml, 2.8 mmol). This was sealed under an atmosphere of CO₂ (ca. 800 psi). The reaction was heated at 100° C. for 16 h, upon which the reaction was cooled to room temperature and vented into a beaker containing EtOAc (50 ml). The cell was rinsed with ethyl acetate and the washings pooled with the vented solution. Silica gel was added (ca 10 g) and the solvent was removed under reduced pressure. Column chromatography of the product (loaded with adsorbed silica, ethyl acetate/hexane 1:4 as eluent) afforded n-butyl-4-nitrocinnamate in 98%.

c) Using the same procedure as in (a) with encapsulated Pd as prepared in Example 1 (20 mg, 5 mmol) and 4-nitrochlorobenzene (175 mg, 1.14 mmol) and heating for 20 h gave n-butyl4-nitrocinnamate (164 mg, 58%) after chromatography.

### EXAMPLE 9

### n-Butyl-4-fluorocinnamate

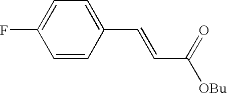

To a stainless steel reactor (10 ml) was added 4-fluorobromobenzene (340 mg, 1.95 mmol), butyl acrylate (0.3 ml, 2.0 mmol), encapsulated Pd resin as prepared in Example 1 (20 mg, 5 mmol) and tetrabutylammonium acetate (800 mg, ca 2.8 mmol). This was sealed under an atmosphere of CO₂ (ca. 800 psi). The reaction was heated at 100° C. for 16 h, upon which the reaction was cooled to room temperature and vented into a beaker containing EtOAc (50 ml). The cell was rinsed with ethyl acetate and the washings pooled with the vented solution. Column chromatography of the product (ethyl acetate/hexane 1:19 as eluent) afforded n-butyl4-fluorocinnamate in 74%.

### EXAMPLE 10

### n-Butyl-4-methoxycinnamate

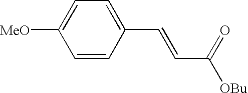

To a stainless steel reactor (10 ml) was added 4-bromoanisole (0.16 ml, 1.30 mmol), butyl acrylate (0.3 ml, 2.0 mmol), encapsulated Pd resin 1 as prepared in Example 1 (20 mg, 5 mmol) and tetrabutylammonium acetate (800 mg, ca 2.8 mmol). This was sealed under an atmosphere of CO₂ (ca. 800 psi). The reaction was heated at 100° C. for 16 h, upon which the reaction was cooled to room temperature and vented into a beaker containing EtOAc (50 ml). The cell was rinsed with ethyl acetate and the washings pooled with the vented solution. Column chromatography of the product (ethyl acetate/hexane 1:19 as eluent) afforded n-butyl4-methoxycinnamate in quantitative yield (300 mg, 1.28 mmol, >99%).

### EXAMPLE 11

### Suzuki Reactions

Suzuki reactions have also been carried out using the same reaction conditions as above. These results are particularly encouraging and a summary of the key results are shown in Table 3.

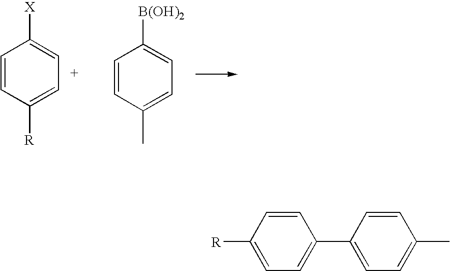

TABLE 3
Suzuki reactions with encapsulated Pd catalyst

| Entry | X | R | Base | Temp° C. | Yield^{a} |
|---|---|---|---|---|---|
| 1 | Br | H | aq. Et₄NOAc | 80 | 60 |
| 2 | Br | H | Bu₄NOAc | 100 | 100 |
| 3 | Br | NO₂ | Bu₄NOAc | 100 | 78^{b} |
| 4 | Cl | NO₂ | Bu₄NOAc | 100 | 60^{b} |
| 5 | Br | F | Bu₄NOAc | 100 | 98^{c} |
| 6 | Br | OMe | Bu₄NOAc | 100 | 60^{b} |

Typical experimental conditions: aryl halide (1 mmol), tolylboronic acid (2 mmol), palladium catalyst (4 mmol) and tetrabutylammonium acetate (2-3 mmol);
^{a}Isolated yield;
^{b}Unoptimised

### EXAMPLE 12

### 4-Methylbiphenyl

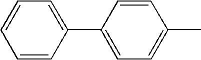

a) To a stainless steel reactor (10 ml) was added 4-bromonitrobenzene (0.1 ml, 0.9 mmol), tolylboronic acid (190 ml, 1.5 mmol), encapsulated Pd resin as prepared in Example 1 (21 mg, 4 mmol) and tetrabutylammonium acetate (600 mg, ca 2 mmol). This was sealed under an atmosphere of CO₂ (ca. 800 psi). The reaction was heated at 100° C. for 16 h with stirring, upon which the reaction was cooled to room temperature and vented into a beaker containing EtOAc (50 ml). The cell was rinsed with ethyl acetate and the washings pooled with the vented solution. Column chromatography of the product (ethyl acetate/hexane 1:99 as eluent) afforded 4-methylbiphenyl in 75% yield. The identical reaction carried out in the absence of stirring gave 4-methylbiphenyl in ca. 45% yield

b) The analogous reaction was carried out using tetraethylammonium hydroxide (2 ml, 2 mmol, 1 M solution in water) afforded 4-methylbiphenyl in 60% yield as calculated from the ¹H NMR spectrum of the crude material.

c) The analogous reaction was carried to (a) out using a larger excess of boronic acid (242 mg, 1.9 mmol) afforded 4-methylbiphenyl in 97% yield after chromatography (hexane as eluent).

### EXAMPLE 13

### 4-Nitro-4′-methylbiphenyl

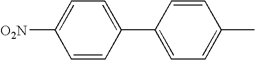

a) To a stainless steel reactor (10 ml) was added 4-bromonitrobenzene (201 mg, 1 mmol), tolylboronic acid (190 ml, 1.5 mmol), encapsulated Pd resin as prepared in Example 1 (20 mg, 4 mmol) and tetrabutylammonium acetate (600 mg, ca 2 mmol). This was sealed under an atmosphere of CO₂ (ca. 800 psi). The reaction was heated at 100° C. for 16 h, upon which the reaction was cooled to room temperature and vented into a beaker containing EtOAc (50 ml). The cell was rinsed with ethyl acetate and the washings pooled with the vented solution. Silica gel was added (ca 10 g) and the solvent was removed under reduced pressure. Column chromatography of the product (ethyl acetate/hexane 1:99 as eluent) afforded 4-nitro-4′-methylbiphenyl in 72%.

b) The analogous reaction was carried out using tetrabutylammonium hydroxide (1 ml, 2 mmol, 1 M solution in water) afforded 4-nitro-4′-methylbiphenyl, after chromatography, in 72% yield.

c) The analogous reaction carried out using 2 eq of tolylboronic acid (270 mg, 2 mmol) afforded 4-nitro-4′-methylbiphenyl in 78% isolated yield.

d) The analogous reaction carried out using 2 eq of tolylboronic acid (270 mg, 2 mmol) and 4-nitrochlorobenzene (165 mg, 1.07 mmol) afforded 4-nitro-4′-methylbiphenyl (137 mg, 0.64 mmol, 60%) after chromatography.

### EXAMPLE 14

### 4-Methy-4′-fluorobiphenyl

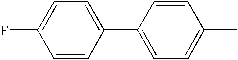

To a stainless steel reactor (10 ml) was added 4-fluorobromobenzene (0.11 ml, 1 mmol), tolylboronic acid (270 ml, 2 mmol), encapsulated Pd resin as prepared in Examples 1 (20 mg, 4 mmol) and tetrabutylammonium acetate (700 mg, ca 2 mmol). This was sealed under an atmosphere of CO₂ (ca. 800 psi). The reaction was heated at 100° C. for 16 h with stirring, upon which the reaction was cooled to room temperature and vented into a beaker containing EtOAc (50 ml). The cell was rinsed with ethyl acetate and the washings pooled with the vented solution. Column chromatography of the product (hexane as eluent) afforded 4-methy-4′-fluorobiphenyl in 100%.

### EXAMPLE 15

### 4-Methy-4′-methoxybiphenyl

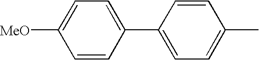

a) To a stainless steel reactor (10 ml) was added 4-bromoanisole (0.12 ml, 0.96 mmol), tolylboronic acid (270 ml, 2 mmol), encapsulated Pd resin 1 (20 mg, 4 mmol) and tetrabutylammonium acetate (800 mg, ca 2.6 mmol). This was sealed under an atmosphere of CO₂ (ca. 800 psi). The reaction was heated at 100° C. for 16 h with stirring, upon which the reaction was cooled to room temperature and vented into a beaker containing EtOAc (50 ml). The cell was rinsed with ethyl acetate and the washings pooled with the vented solution. Column chromatography of the product (hexane as eluent) afforded 4-methy-4′-methoxybiphenyl in 60% (as determined from ¹H NMR of product containing traces of starting material).

b) Heating an analogous reaction for an 40 h afforded 4-methy-4′-methoxybiphenyl in 77% yield.

### EXAMPLE 16

### Stille Reactions

With the success of the above bisphasic conditions, attention was turned to the Stille reaction in which both biphasic and ‘neat’ CO₂ reactions were investigated. A summary of the key results are shown in Table 4. A noteworthy feature of these experiments is that continuous flow experiments are indeed possible, as the products obtained from experiments 2, 4, 6 and 9 are isolated by CO₂ washing. This invention, clearly demonstrates the potential of this methodology as the products are processable under CO₂ systems.

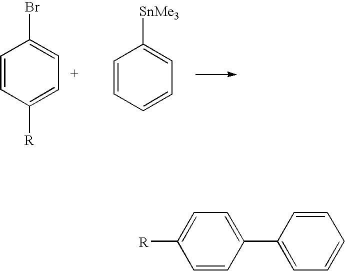

TABLE 4
Stille reactions with encapsulated Pd catalyst

| Entry | X | R | Additive | Temp° C. | Yield^{a} |
|---|---|---|---|---|---|
| 1 | Br | H | Bu₄NOAc | 100 | 58 |
| 2 | B | H | | 100 | 52^{b} |
| 3 | Br^{‡} | NO₂ | Bu₄NOAc | 100 | 60^{b} |
| 4 | Br | NO₂ | | 100 | 34^{b} |
| 5 | Cl | NO₂ | Bu₄NOAc | 100 | 50b |
| 6 | Cl | NO₂ | | 100 | 50^{b} |
| 7 | Br | F | Bu₄NOAc | 100 | 73 |
| 8 | Br | OMe | Bu₄NOAc | 100 | 51^{b} |
| 9 | Br | OMe | | 100 | Similar |

Typical experimental conditions: aryl halide (1 mmol), tolylboronic acid (2 mmol), palladium catalyst (4 mmol) and tetrabutylammonium acetate (2-3 mmol);
^{a}Isolated yield;
^{b}Calculated yield after chromatography, while still retaining unreacted starting material;
^{‡}4-Nitrobiphenyl was difficult to purify from unreacted 4-nitrohalides.

### EXAMPLE 17

### Biphenyl

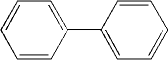

a) To a stainless steel reactor (10 ml) was added 4-bromobenzene (160 mg, 1 mmol), trimethylphenyltin (270 mg, 1.1 mmol), encapsulated Pd resin as prepared in Example 1 (20 mg, 4 mmol) and tetrabutylammonium acetate (800 mg, ca 2.6 mmol). This was sealed under an atmosphere of CO₂ (ca. 800 psi). The reaction was heated at 100° C. for 16 h with stirring, upon which the reaction was cooled to room temperature and vented into a beaker containing EtOAc (50 ml). The cell was rinsed with ethyl acetate and the washings pooled with the vented solution. Column chromatography of the product (hexane as eluent) afforded biphenyl (90 mg, 58%).

b) To a stainless steel reactor (10 ml) was added 4-bromobenzene (682 mg, 4.4 mmol), trimethylphenyltin (344 mg, 1.43 mmol) and encapsulated Pd resin as prepared in Example 1 (40 mg, 10 mmol). This was sealed under an atmosphere of CO₂ (ca. 800 psi). The reaction was heated at 100° C. for 16 h with stirring, upon which the reaction was cooled to room temperature and vented into a round-bottomed flask containing CH₂Cl₂ (50 ml) and silica gel (5 g). Once the cell is vented, it is rinsed (2×) with CO₂. The solvent is evaporated under reduced pressure and the residue chromatographed (dry loaded, silica gel, hexane as eluent) to yield biphenyl (112 mg, 52%).

### EXAMPLE 18

### 4-Nitrobiphenyl

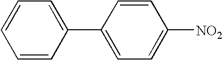

a) To a stainless steel reactor (10 ml) was added 4-bromonitrobenzene (200 mg, 1 mmol), trimethylphenyltin (194 mg, 0.81 mmol), encapsulated Pd resin as prepared in Example 1 (20 mg, 4 mmol) and tetrabutylammonium acetate (800 mg, ca 2.6 mmol). This was sealed under an atmosphere of CO₂ (ca. 800 psi). The reaction was heated at 100° C. for 16 h with stirring, upon which the reaction was cooled to room temperature and vented into a beaker containing EtOAc (50 ml). The product was adsorbed onto silica and was chromatographed (silica gel, ethyl acetate-hexane 1:49 as eluent) and afforded a mixture of 4-nitrobiphenyl and unreacted starting material (128 mg, 90% 4-nitrobiphenyl by ¹H NMR, 60% 4-nitrobiphenyl by calculation).

b) Using the identical approach as in (a), 4-chloronitrobenzene (214 mg, 1.36 mmol), trimethylphenyltin (280 mg, 1.16 mmol), encapsulated Pd resin as prepared in Example 1 (30 mg, 6 mmol) and tetrabutylammonium acetate (800 mg, ca 2.6 mmol) yielded, after chromatography, a mixture of starting material and 4-nitrobiphenyl (174 mg, 75% 4-nitrobiphenyl by integration, 50% 4-nitrobiphenyl by calculation).

c) Using the identical approach as in (a), 4-bromonitrobenzene (387 mg, 1.94 mmol), trimethylphenyltin (324 mg, 1.35 mmol) and encapsulated Pd resin as prepared in Example 1 (20 mg, 5 mmol) yielded, after chromatography, a mixture of starting material and 4-nitrobiphenyl (160 mg, 50% 4-nitrobiphenyl by integration, ca. 34% 4-nitrobiphenyl by calculation)

d) Using the identical approach as in (a), 4-chloronitrobenzene (200 mg, 1.27 mmol), trimethylphenyltin (300 mg, 1.25 mmol) and encapsulated Pd resin as prepared in Example 1 (20 mg, 5 mmol) yielded, after chromatography, a mixture of starting material and 4-nitrobiphenyl (160 mg, 80% 4-nitrobiphenyl by integration, ca. 50% 4-nitrobiphenyl by calculation).

### EXAMPLE 19

### 4-Fluorobiphenyl

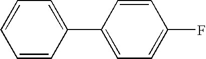

To a stainless steel reactor (10 ml) was added 4-fluorobromobenzene (270 mg, 1.5 mmol), trimethylphenyltin (270 mg, 1.1 mmol), encapsulated Pd resin as prepared in Example 1 (20 mg, 4 mmol) and tetrabutylammonium acetate (700 mg, ca 2 mmol). This was sealed under an atmosphere of CO₂ (ca. 800 psi). The reaction was heated at 100° C. for 16 h with stirring, upon which the reaction was cooled to room temperature and vented into a beaker containing EtOAc (50 ml). The cell was rinsed with ethyl acetate and the washings pooled with the vented solution. Column chromatography of the product (hexane as eluent) afforded 4-fluorobiphenyl (140 mg, 73%).

### EXAMPLE 20

### 4-Methoxybiphenyl

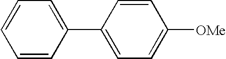

To a stainless steel reactor (10 ml) was added 4-bromoanisole (279 m, 1.5 mmol), trimethylphenyltin (202 mg, 0.84 mmol), encapsulated Pd resin as prepared in Example 1 (20 mg, 5 mmol) and tetrabutylammonium acetate (1 mg, ca 3 mmol). This was sealed under an atmosphere of CO₂ (ca. 800 psi). The reaction was heated at 100° C. for 16 h with stirring, upon which the reaction was cooled to room temperature and vented into a beaker containing EtOAc (50 ml). The cell was rinsed with ethyl acetate and the washings is pooled with the vented solution. Column chromatography of the product (ethyl acetate-hexane 1:49 as eluent) afforded a mixture of 4-methoxybiphenyl in 60% and unreacted starting material (146 mg, 51% 4-methoxybiphenyl calculated after calculated by ¹H NMR).

### EXAMPLE 21

### Continuous Flow Suzuki Reaction with Encapsulated Pd Catalyst

A 50 cm3 stainless steel reactor was fitted with a filter and charged with an encapsulated Pd catalyst (Pd in polyurea, loading of 0.4 mmol/g, 0.125 g, 0.05 mmol). The reactor was then connected to three HPLC injection lines and an exhaust line via a back pressure regulator. The vessel was placed in an oven and was heated to 110° C. CO₂ was charged at a rate of 5 cm³/min until a pressure of 140 kg/cm² (137 bar) was reached. A solution of bromobenzene (0.157 g, 1 mmol), phenylboronic acid (0.122 g, 1 mmol) and tetrabutylammonium actetate (0.301 g, 1 mmol) in methanol (20 cm³) was prepared. The rate of CO₂ addition was adjusted to 2 cm³/min and the reagent solution was added at a rate of 0.1 cm³/min. Once addition was complete methanol (10 cm³) was added at the same rate of 0.1 cm3/min to flush the HPLC line over 1 hr 40. Once this addition was complete the reactor was depressurised. All exhaust from the vessel was vented through ethyl acetate (150 cm³), which was collected, reduced in vacuo and subject to column chromatography on silica gel eluting with 100% isohexane to give the product, biphenyl, as a white crystalline solid (0.014 g, 9%). The reactor was sealed under a CO₂ atmosphere overnight, then the procedure was repeated from step 2. Biphenyl was isolated as before, (0.25 g, 16%) after the first recycle and again (0.24 g, 16%) after the second recycle.

1. A process for metal mediated reactions, particularly cross coupling and carbometallation reactions, wherein the metal is present as a catalyst system comprising a catalyst microencapsulated within a permeable polymer microcapsule shell and the reaction is carried out under super-critical or near super-critical conditions.

2. A process for metal mediated reactions, particularly cross coupling and carbometallation reactions, wherein the metal is present as a catalyst system comprising a catalyst microencapsulated within a permeable polymer microcapsule shell wherein the microcapsule shell is formed by interfacial polymerisation and the reaction is carried out under super-critical or near super-critical conditions.

3. A process for the preparation of optionally substituted biphenyls which comprises reacting an optionally substituted aryl halide or halide equivalent with an optionally substituted aryl boronic acid or ester in the presence of a catalyst system comprising a catalyst microencapsulated within a permeable polymer microcapsule shell under supercritical or near supercritical conditions.

4. A process for the preparation of optionally substituted biphenyls which comprises reacting an optionally substituted aryl halide or halide equivalent with a tri-alkylaryltin in the presence of a catalyst system comprising a catalyst microencapsulated within a permeable polymer microcapsule shell under supercritical or near supercritical conditions.

5. A process for the preparation of optionally substituted alkenes which comprises reacting an optionally substituted aryl halide or halide equivalent with an alkene optionally substituted with up to three substituents in the presence of a catalyst system comprising a catalyst microencapsulated within a permeable polymer microcapsule shell under supercritical or near supercritical conditions.

6. A process for preparation of a hydrogenated product comprising reacting a substrate, wherein the substrate contains a hydrogenatable group or bond, with hydrogen in the presence of a catalyst system comprising a catalyst microencapsulated within a permeable polymer microcapsule shell under supercritical or near supercritical conditions.

7. A process according to any one of claims 1 to 6 wherein supercritical or near supercritical CO₂ is employed.

8. A process according to any one of claims 1 to 6 wherein the permeable polymer microcapsule shell is the product of self-condensation and/or cross-linking of etherified urea-formaldehyde resins or prepolymers in which from about 50 to about 98% of the methylol groups have been etherified with a C₄-C₁₀ alcohol.

9. A process according to any one of claims 1 to 6 wherein the permeable polymer microcapsule shell is a polyurea microcapsule prepared from at least one polyisocyanate and/or tolylene diisocyanate.

10. A process according to claim 9 wherein the polyisocyanates and/or tolylene diisocyanates are selected from the group consisting of 1-chloro-2,4-phenylene diisocyante, m-phenylene diisocyante (and its hydrogenated derivative), p-phenylene diisocyante (and its hydrogenated derivative), 4,4′-methylenebis(phenyl isocyanate), 2,4-tolylene diisocyanate, tolylene diisocyanate (60% 2,4-isomer, 40% 2,6-isomer), 2,6-tolylene diisocyante, 3,3′-dimethyl-4,4′-biphenylene diisocyante, 4,4′-methylenebis (2-methylphenyl isocyanate), 3,3′-dimethoxy-4,4′-biphenylene diisocyanate, 2,2′,5,5′-tetramethyl-4,4′-biphenylene diisocyanate, 80% 2,4- and 20% 2,6-isomer of tolylene diisocyanate, polymethylene polyphenylisocyante (PMPPI), 1,6-hexamethylene diisocyanate, isophorone diisocyanate, tetramethylxylene diisocyanate and 1,5-naphthylene diisocyanate.

11. A process according to any one of claims 1 to 6 wherein the catalyst is a metal catalyst, a metal oxide catalyst, a metal diphosphine catalyst, a metal phosphine catalyst, a metal phosphoramidate catalyst, a metal aminophosphine catalyst, a metal arylamine catalyst, a metal diamine catalyst, a metal aminoalcohol catalyst, a metal phosphate catalyst, a metal salt catalyst, a metal alkoxide catalyst, a metal arene catalyst, a metal arene phosphine catalyst, a metal carbene catalyst, or a metallocycle catalyst.

12. A process according to any one of claims 1 to 6 wherein the catalyst is based on a transition metal.

13. A process according to claim 12 wherein the transition metal on which the catalyst is based is platinum, palladium, osmium, ruthenium, rhodium, iridium, rhenium, scandium, cerium, samarium, yttrium, ytterbium, lutetium, cobalt, titanium, chromium, copper, iron, nickel, manganese, tin, mercury, silver, gold, zinc, vanadium, tungsten or molybdenum.

14. A process according to claim 13 wherein the transition metal on which the catalyst is based is palladium, osmium, ruthenium, rhodium, titanium, vanadium and chromium.

15. A process according to claim 14 wherein the catalyst is colloidal palladium or palladium acetate.

16. A process for the preparation of diols which comprises reacting an olefin in the presence of a catalyst system comprising osmium tetroxide microencapsulated within a permeable polymer microcapsule shell under supercritical or near supercritical conditions
